(19) United States
(12) Patent Application Publication
(10) Pub. No.: US 2009/0297674 A1
(43) Pub. Date: Dec. 3, 2009

(54) Formed meat strip products and methods of making such products

(75) Inventor: Brock Zentz, Andover, KS (US)

Correspondence Address: CARGILL, INCORPORATED, P.O. Box 5624, MINNEAPOLIS, MN, 55440-5624, US

(73) Assignee: CARGILL, INCORPORATED, Wayzata, MN (US)

(21) Appl. No.: 12/454,597

(22) Filed: May 20, 2009

Related U.S. Application Data
(60) Provisional application No. 61/130,352, filed on May 30, 2008.

(51) Int. Cl. B65B 25/06; A23B 4/06; B65D 81/32; G01N 33/12

(52) U.S. Cl. 426/112; 426/392; 426/393; 426/129; 426/232

(57) ABSTRACT

Formed meat strip products and methods of making such meat strip products are described.

### CROSS REFERENCE TO RELATED APPLICATION

This application claims the benefit of Provisional Patent application Ser. No. 61/130,352 filed May 30, 2008 entitled FORMED MEAT STRIP PRODUCTS AND METHODS OF MAKING SUCH PRODUCTS, which is hereby incorporated by reference in its entirety.

### TECHNICAL FIELD

Food products and methods of making such food products are described herein. More particularly, formed meat strip products and methods of making such meat strip products are described.

### BACKGROUND

Meat strip products are used in a variety of applications. In many applications such meat strip products include meat strips formed from ground meat. For example, food products including burritos, fajitas, tacos, steak entrees, steak salads, steak sandwiches and the like include formed meat strip products. Typical meats include beef, pork, chicken, and turkey, for example. Conventional meat strip processing includes grinding the meat and forming the meat into a strip shape with a forming process including a molding step. In such conventional processing, the forming step separately forms each individual meat strip.

### SUMMARY

The meat strip materials described herein are formed into meat blocks, which are subsequently divided into individual meat strips. Forming meat blocks, which are then divided into individual meat strips, provides several advantages. By forming meat blocks that are subsequently divided, throughput at the forming process can be increased as more pounds per forming stroke can be processed. Moreover, by dividing meat blocks, meat strips can be provided with generally straight edges, as compared to meat strips that are individually formed and which have rough edges.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying drawings, which are incorporated in and constitute a part of this application, illustrate several aspects of the invention and together with description of the embodiments serve to explain the principles of the invention. The above mentioned and other advantages of the disclosed meat products and methods, and the manner of attaining them, will become more apparent and the embodiments thereof will be better understood by reference to the following description of the embodiments taken in conjunction with the accompanying drawings, wherein:

FIG. 1 is a schematic flowchart of a prior art process used for forming meat strip products.

FIG. 2 is a photograph showing prior art meat strips formed by the process of FIG. 1.

FIG. 3 is a schematic flowchart of a process for forming meat strips by dividing the meat strips from a meat block.

FIG. 4 is a photograph showing meat strips formed by the process of FIG. 3.

FIG. 5 is a photograph showing a side-by-side comparison, from left to right, of a raw meat strip formed by the process of FIG. 1, a raw meat strip formed by the process of FIG. 3, and a raw whole muscle meat strip.

FIG. 6 is a photograph showing a side-by-side comparison, from left to right, of cooked meat strips formed by the process of FIG. 1, cooked meat strips formed by the process of FIG. 3, and cooked whole muscle meat strips.

### DETAILED DESCRIPTION

The exemplary embodiments described below are not intended to be exhaustive or to limit the embodiments to the precise forms disclosed in the following detailed description. Rather the exemplary embodiments are chosen and described so that others skilled in the art may appreciate and understand the principles and practices of the meat products and processes described herein.

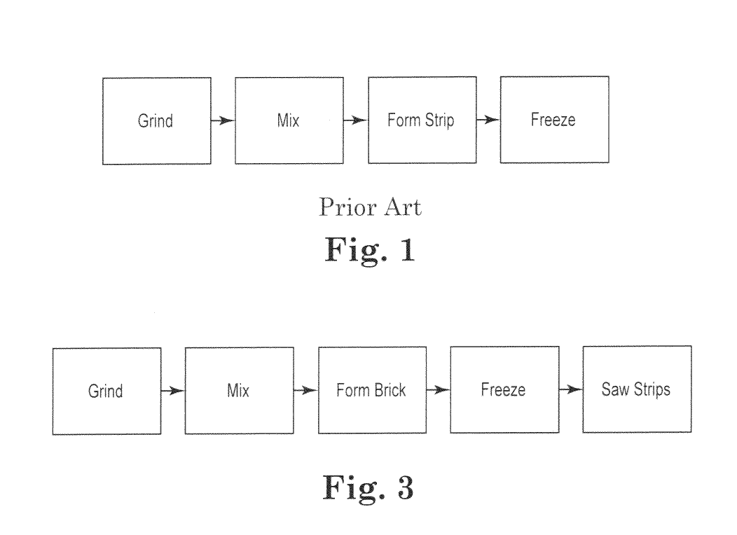

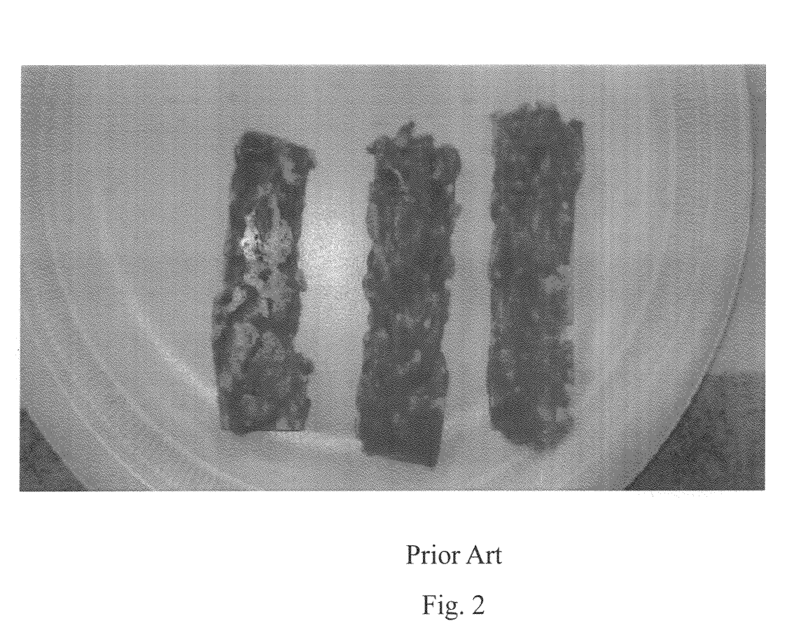

FIG. 1 schematically illustrates a prior art process for forming meat strips where each meat strip is individually formed. The desired meat is ground to a suitable consistency and optionally mixed with marinades, spices, flavor enhancers, and/or tenderizers. A forming process forms the ground meat into meat strips having a desired size and/or weight, after which the meat strips are transferred to a freezing process. Exemplary equipment for forming such meat strips is available from Formax Inc. of Mokena, Ill. (e.g. Formax F6, F12, F26 or equivalent), for example. An exemplary meat strip is about 5 inches long, about 0.5 inches wide, and about 0.5 inches thick and is illustrated in a photograph in FIG. 2. This provides a meat strip that weighs about 0.575 ounces. In an exemplary process for forming such a meat strip, 9.775 ounces are processed per stroke of the forming machine. This provides about 1,649 pounds of meat strips per hour. Meat strips can have any desired dimensions.

In FIG. 3, an exemplary process that forms meat blocks that are subsequently divided into individual meat strips is schematically shown. The meat to be used may be obtained from bovine, porcine, equine, caprine, ovine, avian animals, seafood animals, or any animal commonly slaughtered for food production. Bovine animals include, but are not limited to buffalo, and all cattle, including steers, heifers, cows, and bulls. Porcine animals include, but are not limited to feeder pigs and breeding pigs, including sows, gilts, barrows, and boars. Ovine animals include, but are not limited to, sheep, including ewes, rams, wethers, and lambs. Caprine animals include, but are not limited to, goats, including does, bucks, wethers and kids. Avian animals include, but are not limited to, birds, including chickens, turkeys and ostriches. Seafood animals (including from salt water and freshwater sources) include, but are not limited to, fish and shellfish (such as clams, scallops, shrimp, crabs and lobster).

The desired meat is ground to a suitable consistency and optionally mixed with marinades, spices, flavor enhancers, and/or tenderizers. A forming process forms the ground meat into meat blocks that have a desired size and/or weight and which are sized to be divided into plural meat strips of a desired size and/or weight. In a preferred embodiment, the raw ground meat block is in the shape of a rectangular prism. An exemplary meat block is about 5 inches long, about 0.5 inches wide, and about 25 inches thick. This provides meat blocks that each weigh about 28.75 ounces. Meat blocks having any desired dimensions can be made. After the forming process, the formed meat blocks are at least partially frozen and then divided into meat strips by a dividing process such as a cutting or sawing process. For example, a band saw can be used. The meat strips have a length, width and thickness. For the exemplary meat block described above, fifty meat strips, each about 5 inches long, about 0.5 inches wide and about 0.5 inches thick can be produced from each meat block. Meat strips can have any desired dimensions. Preferred dimensions of the meat strips are from about 4 to 10 inches long, about 0.3 to 2 inches wide and about 0.05 to about 0.8 inches thick. Meat strips of this preferred dimensions have been found to be convenient and desirable for consumption as a snack or as an addition to other food components to form combination food products.

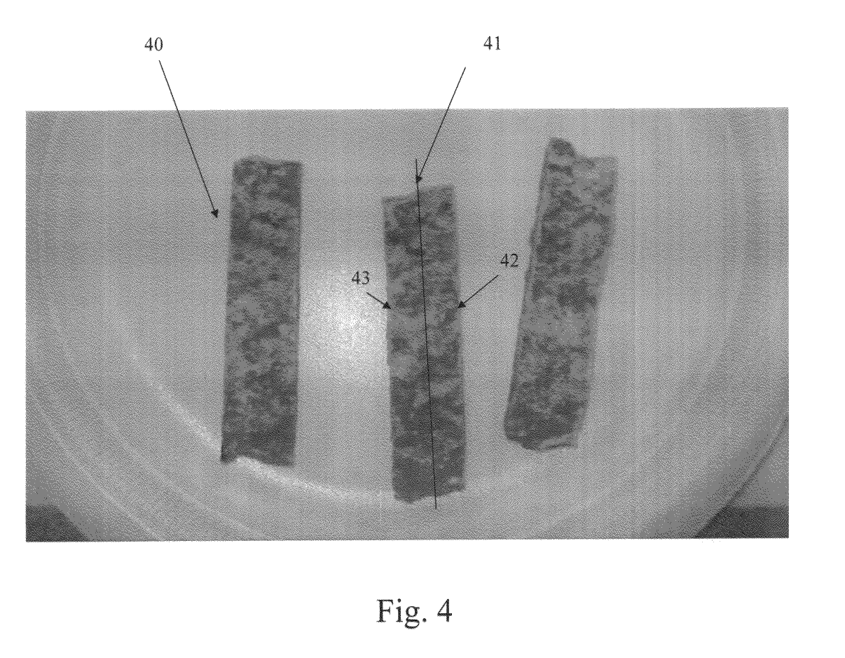

Exemplary meat strips formed by such a process are shown in a photograph in FIG. 4 from a top view. As noted above, the meat strips have a length, width and thickness. Meat strips 40 have a central axis 41 that extends along the length of the strip. In an embodiment, lengthwise edges 42 and 43 are generally parallel to each other and to central axis 41. In an embodiment of the present invention, the meat strips have substantially straight lengthwise edges as viewed from the top of the meat strip portion. By “substantially straight” is meant that the lengthwise edge of the meat strip curves no more than about 10° over a distance of about 5 inches. In another embodiment of the present invention, the lengthwise edges of the meat strips are substantially free from curves, bends or angles. In another embodiment of the present invention, the lengthwise edges of the meat strip are substantially free from deviations greater than 0.2, preferably greater than 0.1 more preferably greater than 0.05 inches in a direction perpendicular to the nominal lengthwise edge. For purposes of the present invention, the nominal lengthwise edge is an imaginary continuous line that defines the edge of the meat strip excluding any gaps, breaks or protrusions which may be present in the meat strip.

In another embodiment of the present invention, the meat strips produced by the present method are substantially uniform in size and/or shape. By “substantially uniform in size” is meant that a test sample group of meat strips has an average size variation from one piece to another of less than 10% by weight, and preferably less than 5% by weight. For purposes of evaluation of the present invention, test sample group is 10 strips selected from an ordinary commercial product run from the same production lot. Preferably, the test sample group is selected from a single package as sent to a consumer. By “substantially uniform in shape” is meant that a test sample group of meat strips has a shape variation in height, width and length from one piece to another of less than 10% in any one dimension, and preferably less than 5% in any one dimension.

In an exemplary process for forming such meat strips from meat blocks, 28.75 ounces are processed per stroke of the forming machine. This provides about 4,851 pounds of meat strips per hour in a single manufacturing line. In a preferred embodiment of the present invention, the method is carried out using automated production equipment to provide substantially uniform meat strips at production speeds greater than 2000 pounds of meat strips per hour, more preferably greater than 3000 pounds of meat strips per hour, and most preferably greater than 4000 pounds of meat strips per hour in a single manufacturing line.

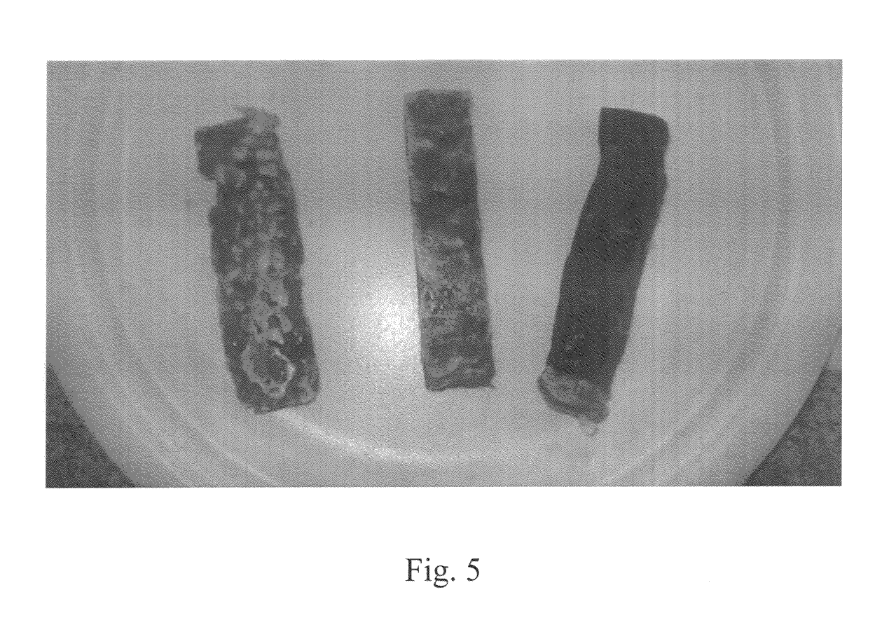

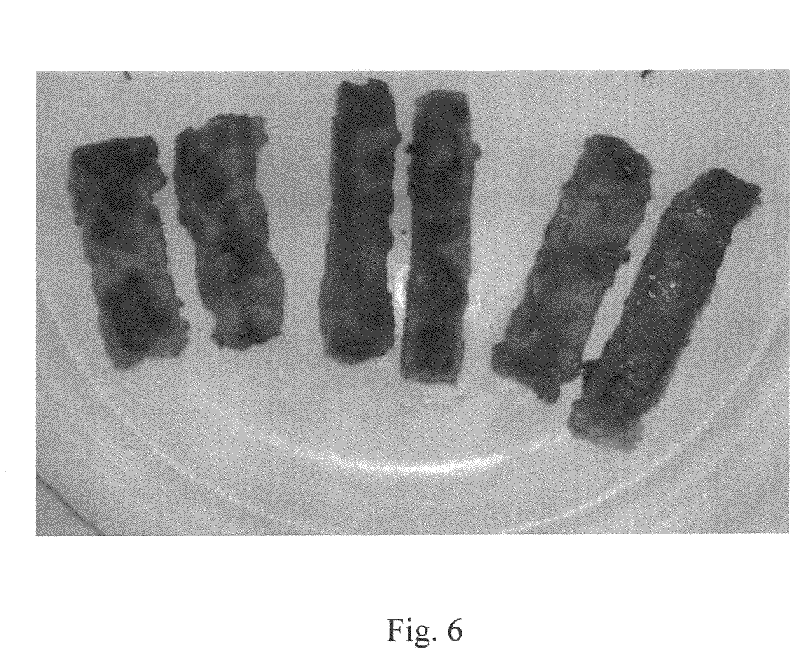

FIG. 5 is a photograph showing, from left to right, a raw meat strip formed individually (also shown in FIG. 2), a raw meat strip divided from a block (also shown in FIG. 4), and a raw whole muscle meat strip. FIG. 6 is a photograph showing, from left to right, cooked meat strips formed individually, cooked meat strips divided from a block, and cooked whole muscle meat strips. As can be seen, meat strips made by dividing the meat strips from a meat block have generally straighter edges than individually formed meat strips.

Meat products and methods of making such meat products have now been described with reference to several embodiments thereof. The entire disclosure of any patent or patent application identified herein is hereby incorporated by reference for all purposes. The foregoing detailed description and examples have been given for clarity of understanding only. No unnecessary limitations are to be understood therefrom. It will be apparent to those skilled in the art that many changes can be made in the embodiments described without departing from the scope of such embodiments. Thus, the scope of the embodiments should not be limited to the meat products and methods described herein, but only by the language of the claims and the equivalents of such language.

What is claimed is:

1. A method of forming meat strips comprising:
providing raw ground meat;
forming the raw ground meat into raw ground meat blocks;
dividing the raw ground meat blocks into raw meat strips after forming the raw ground meat blocks; and
packaging the raw meat strips.

2. The method of claim 1, wherein dividing the raw ground meat blocks comprises sawing the raw ground meat blocks.

3. The method of claim 1, further comprising at least partially freezing the raw ground meat blocks before dividing the raw ground meat blocks.

4. The method of claim 1, wherein providing raw ground meat comprises providing the meat of one or more of bovine, porcine, equine, caprine, ovine, avian animals and seafood animals.

5. The method of claim 1, wherein the raw ground meat block is in the shape of a rectangular prism.

6. The method of claim 1, further comprising cooking the raw meat strips.

7. A meat product made according to claim 1.

8. The meat product of claim 7 in combination with additional ingredients to form a burrito, fajita, taco, steak entree, steak salad, and steak sandwich.

9. A method of forming meat strips comprising:
providing raw ground meat;
forming the raw ground meat into raw ground meat blocks, each raw ground meat block about 5 inches long, about 0.5 inches wide, and about 25 inches thick;
dividing the raw ground meat blocks into fifty raw meat strip portions after forming the raw ground meat blocks, each raw meat strip portion about 0.5 inches thick; and
packaging the raw meat strip portions.

10. The method of claim 9, wherein dividing the raw ground meat blocks comprises sawing the raw ground meat blocks.

11. The method of claim 9, further comprising at least partially freezing the raw ground meat blocks before dividing the raw ground meat blocks.

12. The method of claim 9, wherein providing raw ground meat comprises providing the meat of one or more of bovine, porcine, equine, caprine, ovine, avian animals and seafood animals.

13. The method of claim 9, further comprising cooking the raw meat strips.

14. A meat product made according to claim 9.

15. The meat product of claim 14 in combination with additional ingredients to form a burrito, fajita, taco, steak entree, steak salad, and steak sandwich.

16. The meat product of claim 7, wherein the meat strip has substantially straight lengthwise edges.

17. The meat product of claim 16, wherein the lengthwise edges of the meat strip are substantially free from deviations greater than 0.05 inches in a direction perpendicular to the nominal lengthwise edge.

18. The meat product of claim 7, wherein a test sample group of the meat strips are substantially uniform in size.

19. The meat product of claim 7, wherein a test sample group of the meat strips are substantially uniform in shape.

20. The method of claim 9, wherein the method is carried out using automated production equipment to provide substantially uniform meat strips at production speeds greater than 2000 pounds of meat strips per hour in a single manufacturing line.
